(19) United States
(12) Patent Application Publication
(10) Pub. No.: US 2011/0143451 A1
(43) Pub. Date: Jun. 16, 2011

(54) Method of Operating a Reagent Ion Source

(76) Inventors: John E. P. SYKA, Charlottesville, VA (US); Scott T. Quarmby, Round Rock, TX (US)

(21) Appl. No.: 12/968,177

(22) Filed: Dec. 14, 2010

Related U.S. Application Data
(60) Provisional application No. 61/286,103, filed on Dec. 14, 2009.

(51) Int. Cl. G01N 33/00

(52) U.S. Cl. 436/173

(57) ABSTRACT

A method is disclosed for operating a chemical ionization-type (CI-type) source to generate reagent ions for mass spectrometry experiments, such as electron transfer dissociation (ETD) reagent ions. The method includes periodically reversing current flow in the thermionic filament employed to produce the electron stream. Periodic reversal of the filament current avoids or reduces the problem of carbonaceous growth formation associated with prior art reagent ion sources.

### CROSS-REFERENCE TO RELATED APPLICATION

This application claims the priority benefit under 35 U.S.C. §119(e) of U.S. Provisional Patent Application Ser. No. 61/286,103 for “Extending Lifetime in an Electron Transfer Dissociation (ETD) Reagent Ion Source” filed Dec. 14, 2009, the disclosure of which is incorporated herein by reference.

### BACKGROUND OF THE INVENTION

The present invention relates generally to mass spectrometry, and more particularly to reagent ion sources for electron transfer dissociation (ETD) and other ion-ion reactions.

Mass spectrometry has been extensively employed for ion-ion chemistry experiments, in which analyte ions produced from ionization of a sample substance are reacted with reagent ions of opposite polarity. McLuckey et al. (“Ion/Ion Chemistry of High-Mass Multiply Charged Ions, Mass Spectrometry Reviews, Vol. 17, pp. 369-407 (1998)) discusses various examples of mass spectrometric studies of this type. It has been recently discovered that by selecting an appropriate reagent anion and reacting the reagent anion with a multiply charged analyte cation, a radical site is generated that induces dissociation of the analyte cation into product ions. This process, called electron transfer dissociation (ETD), is described by Hunt et al. in U.S. Pat. No. 7,534,622 for “Electron Transfer Dissociation for Biopolymer Sequence Mass Spectrometric Analysis”, as well as by Syka et al. in “Peptide and Protein Sequence Analysis by Electron Transfer Dissociation Mass Spectrometry”, Proc. Nat. Acad. Sci., vol. 101, no. 26, pp. 9528-9533 (2004) and by Coon et al. in “Anion Dependence in the Partitioning Between Proton and Electron Transfer in Ion/Ion Reactions”, Int. J. Mass Spectrometry, vol. 236, nos. 1-3, pp. 33-42 (2004), all of which are incorporated herein by reference. ETD is a particularly useful tool for proteomics research, since it yields information complementary to that obtained by conventional dissociation techniques (e.g., collisionally induced dissociation), and also because ETD tends to generate product ions having intact post-translational modifications.

Implementation of ETD or other ion-ion experiments in a mass spectrometer requires two ion sources: a first ion source for generating analyte ions from a sample, and a second ion source for generating reagent ions. Commercially available mass spectrometers adapted for ETD generally use a chemical ionization-type (CI-type) ion source to generate the reagent ions. A CI-type ion source is an ion source with a relatively restricted ionization volume gas conductance to the vacuum chamber, such that at relatively modest flows (typically on the order of 0.2 to 4 atm cc/minute) of low molecular weight gases, such as methane or nitrogen, can pressurize the ionization region to about 0.1 to 2 Torr. Such sources typically employ a thermionic filament to produce a stream of electrons, which is directed into the interior of an ionization volume to which molecules of the reagent substance are added in gas phase. The electrons emitted from the filament (emission current range 5-500 μA , most commonly 50 μA) are accelerated to kinetic energies of 50 to 150 eV (typically 70 eV) and once inside the ionization volume, ionize a very high abundance background gas, (typically nitrogen or methane) via electron impact ionization to produce background gas cations and near thermal kinetic energy electrons. The background gas pressure within the ionization volume is generally within the range of 0.05 Torr to 2 Torr, with typical pressures for achieving maximum reagent ion generation in the range of 0.1 to 0.4 Torr. The near thermal electrons are captured by the reagent molecules to produce reagent anions, which are then conveyed to the appropriate region of the mass spectrometer for reaction with analyte ions. Hence ETD reagent anions are generated within a CI-type ion source via electron capture ionization. The background gas pressure within the ionization volume is generally within the range of 0.05 Torr to 2 Torr, with typical pressures for achieving maximum reagent ion generation in the range of 0.1 to 0.4 Torr.

It has been observed that when a CI-type source is employed in the manner described above to generate ETD reagent ions, its filament may exhibit the formation of a carbonanceous growth on the leg adjacent to the negative (−) terminal, resulting in the eventual failure of the filament. It is believed that carbonaceous growth formation arises from the electromigration of dissolved carbon in the filament. As the carbon reaches the cooler portion of the negative leg of the filament, its electromobility drops and the carbon precipitates from solution and accumulates. The primary source of the dissolved carbon is believed to be the reagent molecules, which in the case of ETD reagent molecules, have high carbon content. The applicants believe the reagent molecules decompose upon contact with the filament when the filament is heated to the temperatures necessary to promote electron emission and the resultant carbon atoms are diffused into the bulk filament material. This failure mode is distinguishable from the “normal” filament failure mode, caused by the gradual sublimation of the filament material (e.g., rhenium) at or proximate to the tip (apex) of the filament. The occurrence of carbonaceous growth formation has a strong likelihood of significantly shortening the filament lifetime, thereby increasing instrument downtime due to the need to replace the filament at relatively frequent intervals. The problem of carbonaceous growth formation and consequent filament failure is exacerbated by the conditions at which the CI-type source is typically operated for commonly employed ETD applications. More specifically, the source is operated to provide a “bright” (relatively high ion production rate, usually in excess of 3×10⁷ reagent ions/second) reagent ion source to minimize the time required to generate the requisite numbers of reagent ions, thereby enabling relatively high numbers of ETD MS/MS experiments to be conducted in a given time. Furthermore, in the context of large-scale proteomics studies, the source may be operated on a near-continuous basis over multiple days. The foregoing and other factors may tend to promote and accelerate the processes that produce carbonaceous growth formation.

### SUMMARY

Roughly described, the method of the present invention modifies the operation of a conventional reagent ion source by periodically reversing the direction of current in the thermionic filament. The method includes adding gas-phase organic reagent molecules to an ionization volume, and applying a current to a thermionic filament positioned proximate to the ionization volume. Electrons generated by the filament are directed into the ion volume and interact with the reagent molecules to generate reagent ions. The direction of current flow in the filament is reversed at prescribed intervals. Periodically switching the direction of current flow inhibits the migration of carbon to a location at which significant carbon accumulation occurs, thereby preventing or reducing the rate of carbonaceous growth formation and extending filament lifetime.

### DESCRIPTION OF PREFERRED EMBODIMENTS

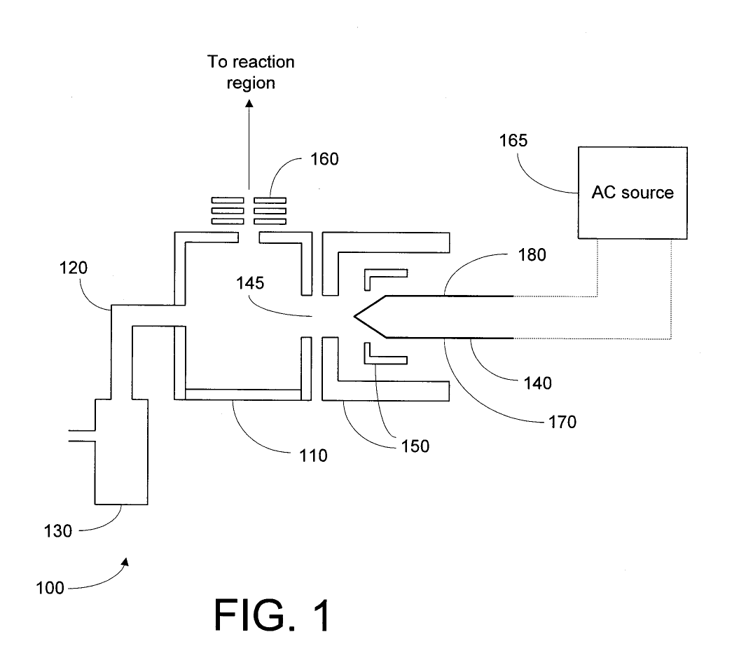

FIG. 1 is a symbolic diagram of an ion source 100 configured to produce reagent anions for ETD. Ion source 100 includes an ionization volume 110 into which reagent molecules are introduced via conduit 120. Ionization volume 110 is located inside a vacuum chamber evacuated to a suitable pressure by a not-illustrated pumping system. The reagent molecules may be generated by controllably heating a vial 130 containing a quantity of the reagent substance in condensed phase form. Fluoranthene is a commonly used ETD reagent, but other organic compounds having suitable electron affinities and properties may be utilized as well. Illustrative examples of compounds useful as ETD reagents are provided in the aforementioned Hunt et al. patent, as well as in PCT Application No. PCT/US2010/047620, which is also incorporated by reference. A flow of a carrier gas transports the reagent molecules from the headspace of vial 130 to the interior of ionization volume 110.

A stream of electrons is generated by passing a current through thermionic filament 140. Filament 140 is typically fabricated from a refractory metal such as rhenium or tungsten (or alloys thereof). Electrons emitted by filament 140 travel (under the influence of an electrical field established by applying suitable potentials to electrodes 150) through aperture 145 into the ionization volume 110 interior. The electron beam may also be guided by a magnetic field established by magnets located behind and on the opposite side of ionization volume 110 from filament 140. The electrons interact (directly or indirectly via production of thermal electrons arising from reaction with background gas molecules, as described above) with molecules of the reagent substance within ion volume 110 to form reagent ions. The reagent anions are extracted from ionization volume 110 by lenses 160, and are transported through an ion guide or other suitable ion optics to an ion trap or other region for reaction with sample cations.

In a conventional ETD source, a static potential is applied across the filament terminals to establish a unidirectional current flow, i.e., toward the filament leg adjacent to the negative terminal. As discussed above, the exposure of filament 140 to the carbon-containing reagent vapor may cause carbonaceous growth formation via migration of dissolved carbon to the cooler portion of the negative leg of filament 140 and its consequent precipitation. It has been observed that the rate of carbonaceous growth formation will depend in part on the abundance of reagent vapor molecules within ionization volume 110, which in turn affects the number of reagent vapor molecules that pass through aperture 145 and contact filament 140. During typical operation, ion volume 110 is filled with the reagent vapor to a partial pressure of at least 1×10⁻⁷ Torr, and preferably at least 3×10⁻⁷ Torr (and in certain preferred implementations at least 1×10⁻⁶ Torr) The rate of carbonaceous growth formation may be reduced by operating the CI source at a lower reagent partial pressure, but doing so will decrease the production of reagent ions, resulting in lower reaction rates/fragmentation efficiencies or lengthened reagent ion fill times.

Embodiments of the present invention employ an alternating current (AC) source 165 to periodically reverse or switch the flow of current in filament 140. Otherwise expressed, current flow is directed toward filament leg 170 during one-half of the AC waveform period and toward opposite filament leg 180 during the other one-half of the waveform period. Reversing the current flow also reverses the direction of electromigration of dissolved carbon. The interval during which current flows in one direction (i.e., one-half of the AC waveform period) should be significantly shorter than a characteristic time required for electromigration of dissolved carbon to a location on a filament leg at which carbon precipitation occurs. This characteristic time will be influenced by a number of factors, including the material and size of filament 140, its operating temperature, and the amplitude of the AC filament current waveform. Testing by the applicant of an ETD reagent source with a rhenium filament of hairpin design, having a diameter of about 0.1 mm and a length of about 5.7 mm, operated under typical conditions revealed that significant reduction in the carbonaceous growth formation rate and extension of filament lifetime were achieved using a sinusoidal AC voltage waveform at a frequency of 50 kHz (20 μs waveform period) having amplitude of ˜1.5 A rms. However, it is believed that beneficial effects may be achieved at significantly lower values of AC waveform frequency (i.e., at significantly longer waveform periods). In certain implementations, the waveform may have a period of several hours or even days.

The waveform applied to filament by AC source 165 may be of any suitable shape, such as sinusoidal, triangular, or square-wave. It is generally preferable that the waveform be symmetrical about the zero potential difference axis, such that electromigration occurs to the same degree, but in different directions, over a full waveform cycle.

While the invention has been described above with reference to its implementation in an ETD reagent ion source, it should not be construed as being limited thereto. Those skilled in the art will recognize that it may be advantageously employed in ion sources configured for production of other types of organic reagent ions, including without limitation proton transfer reagent (PTR) ions , and negative electron transfer dissociation (NETD) reagent ions. Furthermore, the principles of the invention may be applied to other types of ion sources (for example an electron impact or CI source for producing analyte ions in a gas chromatograph/mass spectrometer (GCMS) instrument) in which the filament is exposed to organic molecules in relatively high concentrations.

What is claimed is:

1. A method of generating reagent ions for mass spectrometry experiments, comprising:
applying a potential across a thermionic filament to establish a current flow in the filament and thereby produce a stream of electrons;
adding organic gas-phase reagent molecules to an ionization volume positioned proximate to the filament, such that at least a portion of the reagent molecules interact with the electrons to form reagent ions, wherein the filament is exposed to the reagent molecules; and
periodically reversing the direction of current flow in the filament.

2. The method of claim 1, wherein the reagent molecule is suitable for use as an electron transfer dissociation (ETD) reagent.

3. The method of claim 1, wherein the filament is fabricated from rhenium or an alloy thereof.

4. The method of claim 1, wherein the partial pressure of the reagent molecules in the ionization volume is at least 1×10⁻⁷ Torr.

5. The method of claim 4, wherein the partial pressure of the reagent molecules in the ionization volume is at least 3×10⁻⁷ Torr.

6. The method of claim 5, wherein the partial pressure of the reagent molecules in the ionization volume is at least 1×10⁻⁶ Torr.
